(19) United States
(12) Patent Application Publication
(10) Pub. No.: US 2026/0345205 A1
(43) Pub. Date: Nov. 5, 2026

(54) MAGNETICALLY ACTUATED CRANIAL BIORESORBABLE DISTRACTION SYSTEM

(71) Applicant: Cornell University, Ithaca, NY (US)

(72) Inventors: Mohammed A. Fouda, Ithaca, NY (US); Susan C. Pannullo, Ithaca, NY (US); Caitlin Hoffman, Ithaca, NY (US)

(21) Appl. No.: 19/484,767

(22) PCT Filed: May 15, 2024
(86) PCT No.: PCT/US2024/029432

§ 371 Date: Nov. 14, 2025

Related U.S. Application Data
(60) Provisional application No. 63/466,483, filed on May 15, 2023.

(51) Int. Cl. A61B 17/68; A61B 17/00

(52) U.S. Cl. CPC A61B 17/68; A61B 2017/00367; A61B 2017/00876; A61B 2017/681

(57) ABSTRACT

A magnetically actuated cranial distraction system comprises a bioresorbable distractor device, a guide rod, and a magnetically actuated ratchet. The bioresorbable distractor device is configured to induce cranial bone growth and includes a fixed base member and a mobile base member. The guide rod is coupled with the bioresorbable distractor device and guides movement of the mobile base member relative to the fixed base member. The magnetically actuated ratchet is configured to be magnetically activated from outside a patient to selectively move the mobile base member away from the fixed base member.

### PRIORITY CLAIM

This application claims priority to U.S. Provisional Application Ser. No. 63/466,483 filed 15 May 2023, which is expressly incorporated by reference herein.

### FIELD OF THE DISCLOSURE

The present disclosure relates to a magnetically actuated cranial distraction system for distraction osteogenesis.

### BACKGROUND

Distraction osteogenesis is a process used to repair skeletal deformities. The process includes slowly separating bone and allowing new bone to form in the gap between the separated bone segments. In some cases, distraction osteogenesis using traditional systems involves risks, such as infection and system failure. There is a need for improved systems for use in distraction osteogenesis.

### SUMMARY

According to the present disclosure, a magnetically actuated cranial distraction system for distraction osteogenesis is described. The magnetically actuated cranial distraction system is configured to induce cranial bone growth by separating cranial bone segments to induce growth therebetween.

In some embodiments, the magnetically actuated cranial distraction system includes a bioresorbable distractor device, a guide rod, and a magnetically actuated ratchet. The bioresorbable distractor device is fixed to the cranial bone segments. The guide rod selectively moves in response to a magnetic force applied to the magnetically actuated ratchet. As the guide rod translates, a mobile base member of the bioresorbable distractor device translates with the guide rod away from a fixed base member of the bioresorbable distractor device to separate the cranial bone segments.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIG. 1 is a front perspective view of a magnetically actuated cranial distraction system including a bioresorbable distractor device configured to be fixed to cranial bone segments and having a fixed base member and a mobile base member, a guide rod extending through the fixed base member and the mobile base member, and a magnetically actuated ratchet configured to selectively move the mobile base member axially away from the fixed base member to induce cranial bone growth between separated cranial bone segments;

FIG. 2 is an exploded view of the magnetically actuated cranial distraction system of FIG. 1 showing the magnetically actuated ratchet includes an activation lever having a ferromagnetic component therein and configured to rotate in response to a magnetic force, a guide barrel, and a connection member, the guide rod extends through each of the activation lever and the guide barrel, and the connection member extends between the activation lever and the guide barrel to transfer rotational energy from the activation lever to the guide barrel to cause the guide barrel to rotate with the activation lever;

FIG. 3 is a side perspective view of the magnetically actuated cranial distraction system of FIG. 1 with a cover of the magnetically actuated ratchet removed and showing that the connection member wraps around and interconnects the activation lever and the guide barrel to selectively couple the activation lever and the guide barrel together;

FIG. 4 is a side perspective view of the magnetically actuated cranial distraction system of FIG. 1 with the cover of the magnetically actuated ratchet removed and showing that the activation lever is formed to include a rotational blocker extending outwardly away from the guide rod and configured to engage the fixed base member to block over rotation of the activation lever about the axis;

FIG. 5 is a perspective view of an external activator included in the magnetically actuated cranial distraction system and including a body, an external magnetic component inside of the body, and a plunger coupled to the external magnetic component, the external magnetic component is configured to apply the magnetic force to the activation lever while being in close proximity to the ferromagnetic component of the activation lever so that the activation lever rotates;

FIG. 6 is a cross-section view of the external activator of FIG. 5 showing that the body defines a cavity therein and suggesting that the external magnetic component is arranged in the cavity to move within the cavity with the plunger;

FIG. 7 is a perspective view of a guide helmet included in the magnetically actuated cranial distraction system and configured to be placed on a head of a patient, the guide helmet formed to include an opening that is aligned with the implanted magnetically actuated ratchet so that the external activator can be aligned with the activation lever of the magnetically actuated ratchet to apply the magnetic force thereto;

FIG. 8 is a perspective view of a measuring gauge included in the magnetically actuated cranial distraction system and configured to be used to indicate the distance between the fixed base member and the mobile base member;

FIG. 9 is a perspective view of an alternative embodiment of a magnetically actuated cranial distraction system, the magnetically actuated cranial distraction system including a bioresorbable distractor device having a first base member and a second base member, a guide rod extending through the first base member and coupled with the second base member, and a distractor activation assembly configured to selectively move the second base member axially away from the first base member;

FIG. 10 is a perspective view of an alternative embodiment of a magnetically actuated cranial distraction system, the magnetically actuated cranial distraction system including a bioresorbable distractor device having a first base member and a second base member, a guide rod extending through the first base member and coupled with the second base member, and a distractor activation assembly configured to selectively move the second base member axially away from the first base member;

FIG. 11 is a perspective view of an alternative embodiment of a magnetically actuated cranial distraction system, the magnetically actuated cranial distraction system including a bioresorbable distractor device having a first base member and a second base member, a guide rod extending through the first base member and coupled with the second base member, and a distractor activation assembly configured to selectively move the second base member axially away from the first base member;

FIG. 12 is a perspective view of an alternative embodiment of a magnetically actuated cranial distraction system similar to the magnetically actuated cranial distraction system of FIG. 11, the magnetically actuated cranial distraction system including a bioresorbable distractor device having a first base member and a second base member, a guide rod extending through the first base member and coupled with the second base member, and a distractor activation assembly having an opposite orientation than the distractor activation assembly of FIG. 11;

FIG. 13 is a perspective view of an alternative embodiment of a magnetically actuated cranial distraction system, the magnetically actuated cranial distraction system including a bioresorbable distractor device having a first base member and a second base member, a guide rod extending through the first base member and coupled with the second base member, and a distractor activation assembly configured to selectively move the second base member axially away from the first base member;

FIG. 14 is a perspective view of an alternative embodiment of a magnetically actuated cranial distraction system similar to the magnetically actuated cranial distraction system of FIG. 13, the magnetically actuated cranial distraction system including a bioresorbable distractor device having a first base member and a second base member, a guide rod extending through the first base member and coupled with the second base member, and a distractor activation assembly having an opposite orientation than the distractor activation assembly of FIG. 13; and

FIG. 15 is a perspective view of an alternative embodiment of a magnetically actuated cranial distraction system, the magnetically actuated cranial distraction system including a bioresorbable distractor device having a first base member and a second base member, a guide rod extending through the first base member and coupled with the second base member, and a distractor activation assembly configured to selectively move the second base member axially away from the first base member and having a relatively large activation lever.

### DETAILED DESCRIPTION

The present disclosure relates to a magnetically actuated cranial distraction system 10 for distraction osteogenesis. Cranio-maxillo-facial conditions, such as syndromic craniosynostosis, comprise a premature fusion of one or more cranial sutures and/or sutures of the midface and skull base. Distraction osteogenesis is a biological and mechanical process that promotes bone growth between bone segments by pulling the bone segments apart to allow new bone to fill the gap between the bone segments.

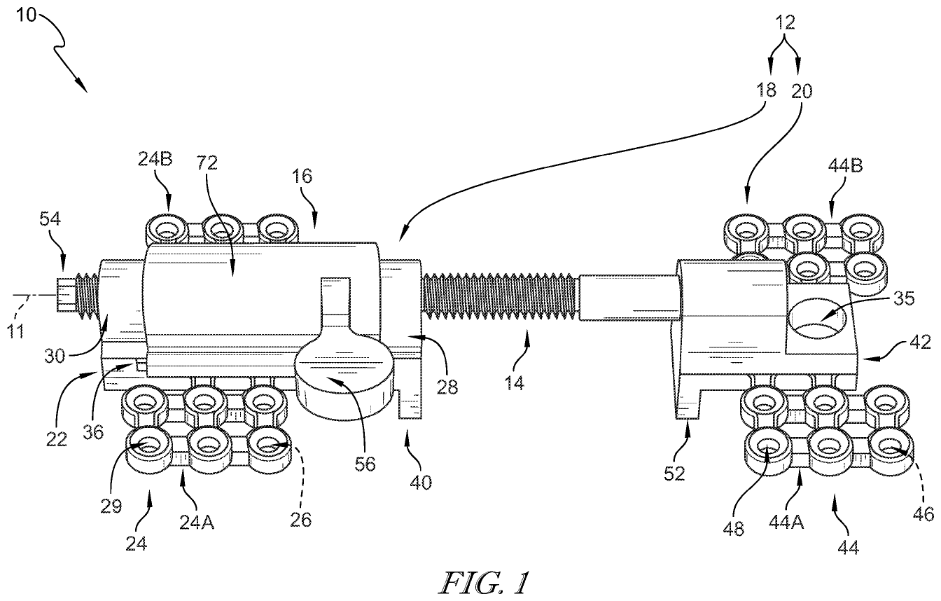

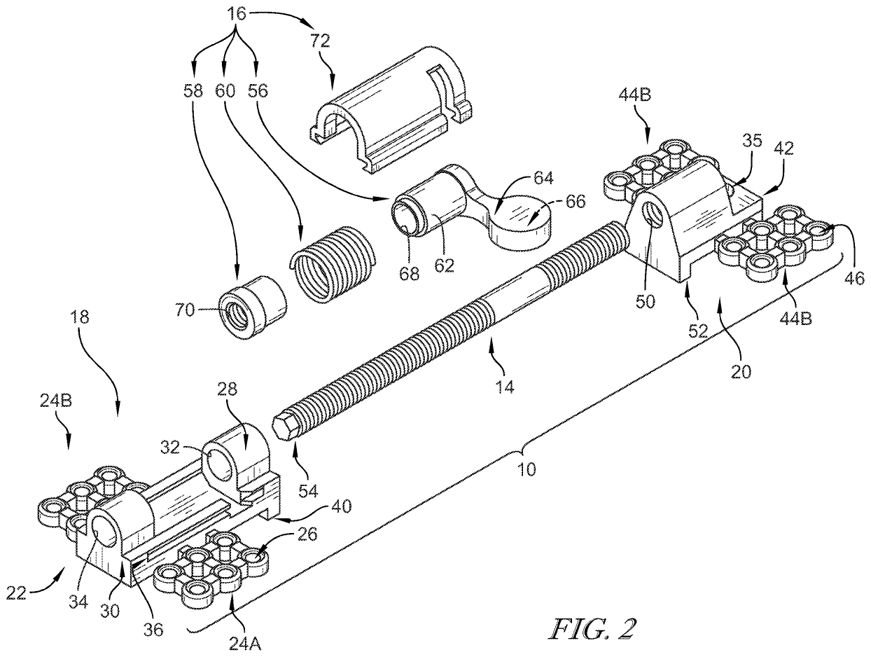

The magnetically actuated cranial distraction system 10 includes a bioresorbable distractor device 12, a guide rod 14, and a magnetically actuated ratchet 16, as shown in FIGS. 1 and 2. The bioresorbable distractor device 12 is configured to induce cranial bone growth. The bioresorbable distractor device 12, which may also be referred to as a distractor device 12, includes a fixed base member 18 and a mobile base member 20, each attached to cranial bone segments adjacent one another relative to an axis 11. During surgery, an osteotomy of the cranial bone is created (in other words, a suture line), and the magnetically actuated cranial distraction system 10 is fit to the cranial bone. For example, the fixed base member 18 is fixed to the cranial bone on one side of the suture line (i.e., one cranial bone segment), and the mobile base member 20 is fixed to the cranial bone on the other side of the suture line (i.e., an adjacent cranial bone segment). During implantation, the fixed base member 18 and the mobile base member 20 abut one another. The magnetically actuated cranial distraction system 10 is then used to gradually separate the cranial bone segments so that new bone can form in the gap therebetween. As the cranial bone segments are separated, the fixed base member 18 and the mobile base member 20 gradually grow farther apart from one another, as shown in FIG. 1.

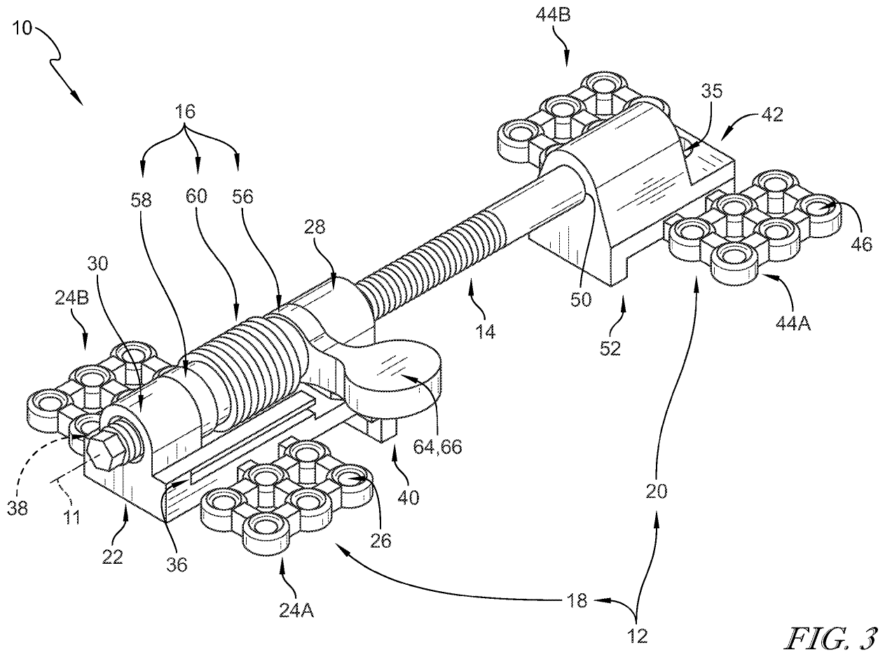

The guide rod 14 extends between and interconnects the fixed base member 18 and the mobile base member 20, as shown in FIGS. 1 and 3. The guide rod 14 is configured to selectively move along the axis 11 relative to the fixed base member 18 to move the mobile base member 20 axially away from the fixed base member 18. In doing so, the cranial bone segments that the base members 18, 20 are fixed to are separated so that new bone can form in the gap therebetween.

The magnetically actuated ratchet 16 is configured to selectively move the guide rod 14, and thus, the mobile base member 20 along the axis 11 relative to the fixed base member 18. Each of the bioresorbable distractor device 12, the guide rod 14, and the magnetically actuated ratchet 16 are entirely located underneath skin of a patient. The axial movement of the guide rod 14 and the mobile base member 20 is activated via a magnetic force applied to the magnetically actuated ratchet 16. The magnetic force is applied from an area exterior to the skin of the patient.

Generally, traditional distractor systems require an external activation port protruding through the skin of the patient that is used to separate bone segments. These distractor systems come with many drawbacks. For example, infection may be common due to the protrusion of the system through the skin. As another example, traditional distractor systems may lead to cerebrospinal fluid (CSF) leak. Additionally, a second procedure is necessary to remove the distractor system from the patient, which comes with associated risks of anesthesia, blood loss, and infection, in addition to the added costs of another surgical intervention.

Each of the bioresorbable distractor device 12, the guide rod 14, and the magnetically actuated ratchet 16 are entirely located underneath the skin of the patient, and the axial movement of the guide rod 14 and the mobile base member 20 is activated via the magnetic force applied to the magnetically actuated ratchet 16 from the area exterior to the skin. In this way, the magnetically actuated cranial distraction system 10 does not require an external activation port or any protrusion through the skin such that there is no open wound created by the magnetically actuated cranial distraction system 10.

Turning back to the bioresorbable distractor device 12, the fixed base member 18 of the device 12, which may be referred to as a first base member 18, includes a base 22, a plate 24, and a plurality of fasteners 26, as shown in FIGS. 1-4. Illustratively, the plate 24 includes a first plate 24A and a second plate 24B. The first plate 24A extends outwardly away from the base 22 from a first side thereof, and the second plate 24B extends outwardly away from the base 22 from a second side thereof opposite the first side. Each plate 24A, 24B is formed to include a plurality of fastener-receiver holes 29 extending therethrough to receive the plurality of fasteners 26 therein. In some embodiments, each plate 24A, 24B is shaped to define a honeycomb-like geometry to allow for bending of the plate 24A, 24B during implantation to match the cranial bone segment geometry. Each of the plurality of fasteners 26 extends through a corresponding one of the plurality of fastener-receiver holes 29 and into the cranial bone segment to fix the fixed base member 18 to the cranial bone segment.

Illustratively, six fasteners 26 are used per plate 24A, 24B, though any number of fasteners 26 may be used. The plurality of fasteners 26 includes, for example, bioresorbable ultrasound-activated pins (UAPs), bioresorbable heat-activated pins (HAPs), bioresorbable screws, bone screws, or any other suitable fastener. UAPs and HAPs allow for attachment to thinner bone segments because tapping and/or threading is not required. UAPs fuse with the plates 24A, 24B, increasing the fixation strength. In use, a UAP is inserted into each of the plurality of fastener-receiver holes 29. Ultrasound is applied, which allows the UAPs to liquefy and flow into a drilled hole in the cranial bone segment underneath. After solidification, the UAPs are fixed to the cranial bone segment and the corresponding plate 24A, 24B.

The fixed base member 18 further includes a first support bracket 28 and a second support bracket 30, as shown in FIGS. 1-4. The first support bracket 28 and the second support bracket 30 extend upwardly away from the base 22. The first support bracket 28 and the second support bracket 30 are spaced apart from one another, and the first support bracket 28 is located between the second support bracket 30 and the mobile base member 20. The first support bracket 28 is formed to include a first through hole 32 extending axially therethrough, and the second support bracket 30 is formed to include a second through hole 34 extending axially therethrough. Illustratively, each of the through holes 32, 34 are not threaded.

The base 22 is formed to include a first groove 36 on the first side thereof and a second groove 38 on the second side thereof opposite the first side, as shown in FIGS. 1-4. The base 22 is further formed to include a lip 40 extending downwardly away from the first support bracket 28. The lip 40 is configured to position the fixed base member 18 relative to the cranial bone segment while the fixed base member 18 is being fixed to the cranial bone segment. The lip 40 hangs over an edge of the cranial bone segment where the cut was made during surgery. The lip 40 relieves the plurality of fasteners 26 from shear forces that occur during the use of the system 10.

The mobile base member 20 of the device 12, which may be referred to as a second base member 20, includes a base 42, a plate 44, and a plurality of fasteners 46, as shown in FIGS. 1-4. Illustratively, the plate 44 includes a first plate 44A and a second plate 44B. The first plate 44A extends outwardly away from the base 42 from a first side thereof, and the second plate 44B extends outwardly away from the base 42 from a second side thereof opposite the first side. Each plate 44A, 44B is formed to include a plurality of fastener-receiver holes 48 extending therethrough to receive the plurality of fasteners 46 therein. In some embodiments, each plate 44A, 44B is shaped to define a honeycomb-like geometry to allow for bending of the plate 44A, 44B to match the cranial bone segment geometry. Each of the plurality of fasteners 46 extends through a corresponding one of the plurality of fastener-receiver holes 48 and into the cranial bone segment to fix the mobile base member 20 to the cranial bone segment. Illustratively, the plurality of fasteners 46 is the same as the plurality of fasteners 26 such that the description of the plurality of fasteners 26 applies to the plurality of fasteners 46.

The base 42 of the mobile base member 20 is formed to include a threaded hole 50 to receive the guide rod 14 therein, as suggested in FIG. 2. The threaded hole 50 is aligned with the first through hole 32 and the second through hole 34 of the fixed base member 18 about the axis 11. The guide rod 14 is fixed to the mobile base member 20 via locking threads of the threaded hole 50 so that the mobile base member 20 and the guide rod 14 axially translate along the axis 11 with one another.

The mobile base member 20 is formed to include a lip 52 extending downwardly away from the base 42. The lip 52 is configured to position the mobile base member 20 relative to the cranial bone segment while the mobile base member 20 is being fixed to the cranial bone segment. The lip 52 hangs over an edge of the cranial bone segment where the cut was made during surgery. The lip 52 relieves the plurality of fasteners 46 from shear forces that occur during the use of the system 10.

The guide rod 14, which may be referred to as a threaded rod 14, extends through each of the second through hole 34 of the second support bracket 30 of the fixed base member 18, the first through hole 32 of the first support bracket 28 of the fixed base member 18, and the threaded hole 50 of the mobile base member 20, as shown in FIG. 1. Illustratively, the guide rod 14 is formed to include two threaded portions and a non-threaded portion located between the two threaded portions, as shown in FIG. 2. One of the threaded portions mates with the threads of the threaded hole 50. The non-threaded portion of the guide rod 14 helps to maintain the structural integrity of the guide rod 14 at a point with the greatest bending moment and likelihood of failure. In some embodiments, the guide rod 14 is entirely non-threaded. In some embodiments, the guide rod 14 is entirely threaded.

An end of the guide rod 14 adjacent the fixed base member 18 is shaped to include a hexagonal head 54, as shown in FIGS. 1 and 2. The hexagonal head 54 allows for tightening of the guide rod 14 into the threaded hole 50 of the mobile base member 20 via a hex-socket or a hex-head wrench. The length of the guide rod 14 is chosen based on the extent of distraction needed for the particular patient.

The magnetically actuated ratchet 16, which may be referred to as a distractor activation assembly 16, includes an activation lever 56, a guide barrel 58, and a connection member 60, as shown in FIGS. 2 and 3. The activation lever 56 rotates about the axis 11 in response to the magnetic force. The guide barrel 58, which may also be referred to as a threaded barrel 58, is located axially between the second support bracket 30 and the activation lever 56, and the activation lever 56 is located axially between the guide barrel 58 and the first support bracket 28. The connection member 60 extends around the activation lever 56 and the guide barrel 58, as shown in FIGS. 3 and 4.

The activation lever 56 includes a lever barrel 62, a lever arm 64, and a ferromagnetic component 66, as shown in FIG. 3. The lever barrel 62 is formed to include a hole 68 extending axially therethrough to receive the guide rod 14 therein. Illustratively, the hole 68 is formed without threads. The lever arm 64 extends outwardly away from the lever barrel 62. The ferromagnetic component 66 is arranged inside the lever arm 64 so that the ferromagnetic component 66 is spaced apart from the axis 11. The activation lever 56 is further formed to include a rotational blocker 65 extending away from the lever barrel 62 in a direction opposite the lever arm 64. The rotational blocker 65 is configured to engage the fixed base member 18 to block over rotation of the activation lever 56 about the axis 11.

The guide barrel 58 is formed to include a threaded hole 70 extending axially therethrough, as shown in FIG. 2. The threaded hole 70 is aligned with each of the through holes 32, 34 of the fixed base member 18 and the hole 68 of the lever barrel 62. The guide rod 14 extends through and mates with the threaded hole 70 as suggested in FIG. 2. A male notch of the lever barrel 62 mates with a female notch of the guide barrel 58 to maintain the alignment of the barrels 58, 62.

The connection member 60 is illustratively a wrap spring 60, as shown in FIG. 2. The wrap spring 60 extends around the guide barrel 58 and the lever barrel 62. During assembly, the wrap spring 60 is positioned on one of the guide barrel 58 and the lever barrel 62, and the guide barrel 58 and the lever barrel 62 are aligned with one another such that the wrap spring 60 extends around each of the guide barrel 58 and the lever barrel 62. The assembled guide barrel 58 and the lever barrel 62 are positioned between the first support bracket 28 and the second support bracket 30. The guide rod 14 slides through the second through hole 34 of the second support bracket 30, is threaded through the threaded hole 70 of the guide barrel 58, slides through the hole 68 of the lever barrel 62, and slides through the first through hole 32 of the first support bracket 28. The guide rod 14 is then threaded through the threaded hole 50 of the mobile base member 20 until the guide rod 14 is locked with the mobile base member 20.

In some embodiments, the magnetically actuated ratchet 16 further includes a cover 72, as shown in FIGS. 1 and 2. The cover 72 is positioned over the guide barrel 58, the connection member 60, and the lever barrel 62 and snaps into place via the grooves 36, 38 formed in the base 22 of the fixed base member 18. The cover 72 is configured to protect the components underneath from tissue granulation.

During use, an external magnetic component 74 is positioned near the ferromagnetic component 66 in the lever arm 64. The ferromagnetic component 66 is located underneath the skin of the patient, while the external magnetic component 74 is located exterior to the skin of the patient. The external magnetic component 74 is aligned with the ferromagnetic component 66. In doing so, the external magnetic component 74 exerts the magnetic force on the ferromagnetic component 66 in the lever arm 64. The magnetic force causes the lever arm 64 to rise (i.e., rotate about the axis 11) toward the external magnetic component 74. The lever barrel 62, thus, rotates about the axis 11 with the lever arm 64. Friction between the lever barrel 62 and the wrap spring 60 as the lever barrel 62 rotates causes the wrap spring 60 to tighten around the lever barrel 62 and the guide barrel 58. While the wrap spring 60 is tightened, the wrap spring 60 couples the barrels 58, 62 together such that the wrap spring 60 transfers rotational energy from the lever barrel 62 to the guide barrel 58 to cause the guide barrel 58 to rotate about the axis 11 with the lever barrel 62 in a first direction. As the guide barrel 58 rotates, the guide rod 14 continues to be threaded through the threaded hole 70 of the guide barrel 58. In other words, the guide rod 14 moves axially through the guide barrel 58. The rotation of the guide barrel 58 translates into axial movement of the guide rod 14. Because the guide rod 14 is fixed to the mobile base member 20, as the guide rod 14 axially translates, the mobile base member 20 axially translates therewith to increase a distance between the base members 18, 20. As the mobile base member 20 moves axially away from the fixed base member 18, small, discrete distractions are made.

As the external magnetic component 74 moves away from the lever arm 64 and the ferromagnetic component 66, the lever arm 64 lowers, and the lever barrel 62 rotates back (in a second direction opposite the first direction) to the initial orientation of the lever barrel 62 prior to the application of the magnetic force. In some embodiments, the lever arm 64 assumes the initial orientation due to gravity. In some embodiments, the lever arm 64 assumes the initial orientation due to the elasticity of the skin. The wrap spring 60 loosens, thereby allowing the guide barrel 58 and the lever barrel 62 to rotate freely from one another. Friction caused by the force of the guide barrel 58 on the second support bracket 30 of the fixed base member 18 prevents the guide barrel 58 from rotating back to the initial orientation of the guide barrel 58 while the lever barrel 62 returns to the initial orientation.

The base members 18, 20 are formed of bioresorbable materials. For example, the base members 18, 20 may be formed of polymers such as polylactides (PLAs), polydioxanones (PDOs), polycaprolactones (PCLs), poly L-lactic acid (PLLA), polyglycolic acid (PGA), and polydioxanone. As another example, the base members 18, 20 may be formed of copolymers such as 82:18 L-lactic acid: glycolic acid, self-reinforcing lactic acid/glycolic acid, self-reinforcing 70:30 L-lactic acid: DL-lactic acid, 70:30 lactic acid L/DL. In some embodiments, the base members 18, 20 may be formed of polymethyl methacrylate (PMMA). In illustrative embodiments, the base members 18, 20 may be formed of a magnesium-based alloy of magnesium, gallium, and zinc. Magnesium-based alloys of magnesium, gallium, and zinc have relatively high strength, are biodegradable, promote bone growth, have antimicrobial properties, are corrosion resistant, and have a bone-like modulus of elasticity. The degradation time of the magnesium-based alloy can be tailored to match the period required for bone distraction and healing of the patient. In some embodiments, to delay degradation of the magnesium-based alloy, the magnesium-based alloy may undergo purification, an alloying treatment, and/or a surface coating. The surface treatment may comprise anodization for improved surface hardness. In some embodiments, the base members 18, 20 may comprise magnesium-based metal matrix composites. The body resorbs the materials of the base members 18, 20, which eliminates the need for a removal surgery.

The guide rod 14 and the magnetically actuated ratchet 16 are formed of biocompatible materials. The guide rod 14 and the magnetically actuated ratchet 16 are more accessible to remove from the patient than the base members 18, 20 because these components are not fixed to the cranial bone segments like the base members 18, 20. Due to the ease of removal, the guide rod 14 and the magnetically actuated ratchet 16 need not be formed of bioresorbable materials. However, in some embodiments, the guide rod 14 and the magnetically actuated ratchet 16 may be formed of bioresorbable materials. In some embodiments, the guide rod 14 and the magnetically actuated ratchet 16 (not including the ferromagnetic component 66) are formed of titanium-6-aluminum-4-vanadium (Ti—6Al—4V). This material has a relatively high strength, is corrosion resistant, and is biocompatible. In some embodiments, the material undergoes surface treatments, such as passivation to enhance corrosion resistance, anodization to improve surface hardness and wear resistance, and/or surface texturing or coating with hydroxyapatite to promote osseointegration.

In some embodiments, the ferromagnetic component 66 is formed of samarium-cobalt (SmCo). This material offers high magnetic strength and thermal stability. In some embodiments, the material is coated with parylene or epoxy to provide corrosion protection to the ferromagnetic component 66. In some embodiments, a high-permeability material, such as Mu-metal, may be used to shield the magnetic field of the ferromagnetic component 66 so that the magnetic field does not interfere with surrounding tissues and/or other medical devices.

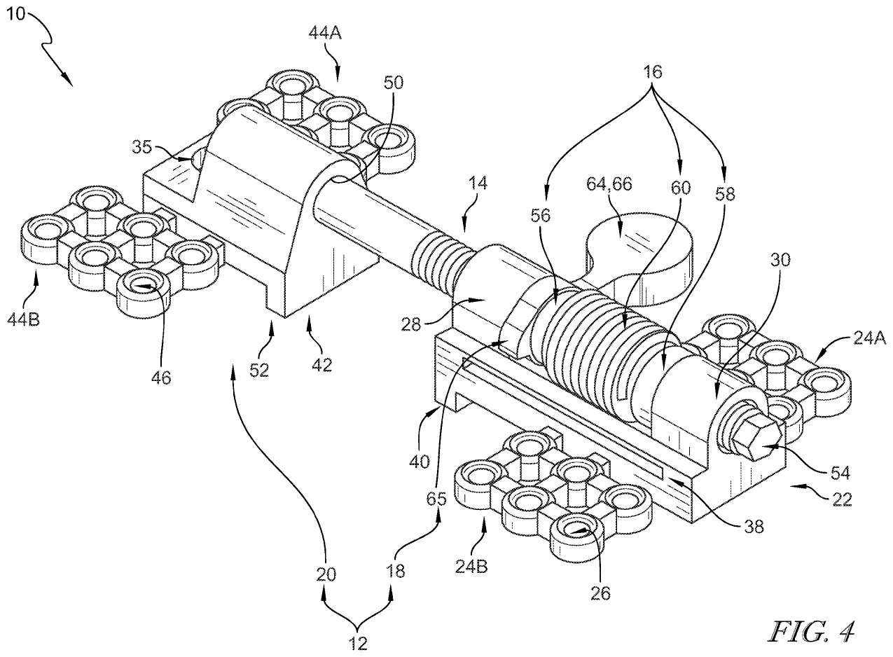

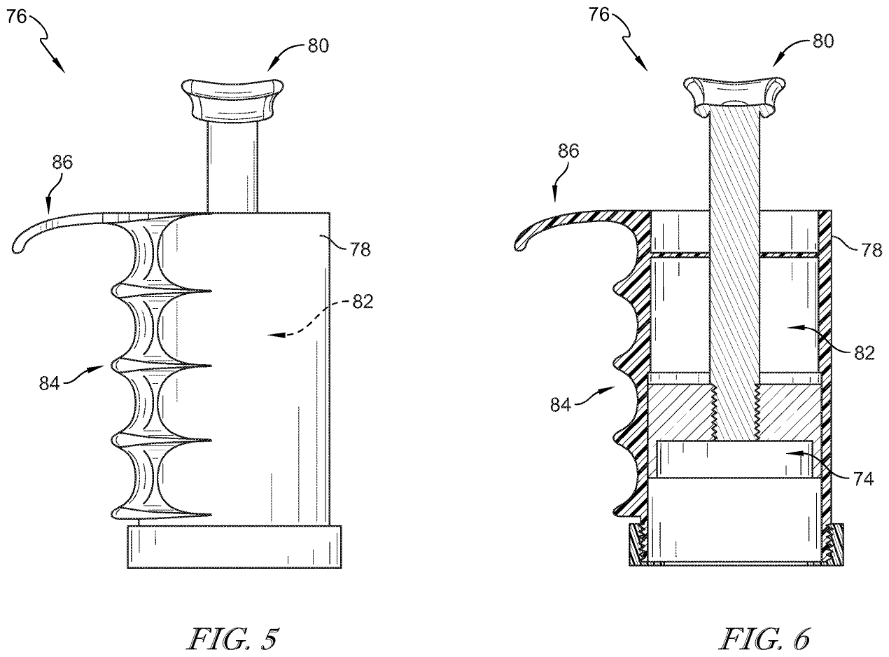

In some embodiments, the system 10 includes an external activator 76 that houses the external magnetic component 74 therein, as shown in FIGS. 5 and 6. The external activator 76 includes a body 78 and a plunger 80. The body 78 defines a chamber 82 therein, and the external magnetic component 74 is located in the chamber 82. The plunger 80 is coupled with the external magnetic component 74. In response to a downward force on the plunger 80, the external magnetic component 74 moves downward within the chamber 82 toward an end of the external activator 76. In use, the external activator 76 is positioned near the lever arm 64 of the magnetically actuated ratchet 16, and the plunger 80 is pushed downwardly via the downward force so that the external magnetic component 74 moves to the end of the external activator 76 to be adjacent the ferromagnetic component 66 in the lever arm 64. When the external magnetic component 74 is positioned at the end of the external activator 76, the external magnetic component 74 applies the magnetic force to the ferromagnetic component 66. The plunger 80 is spring-loaded such that the plunger 80 may be pulled upwardly after applying the magnetic force to move the external magnetic component 74 away from the end of the external activator 76. In some embodiments, the external activator 76 includes a hand grip 84 formed on the body 78 and a handle 86 extending away from the body 78 for ease of use.

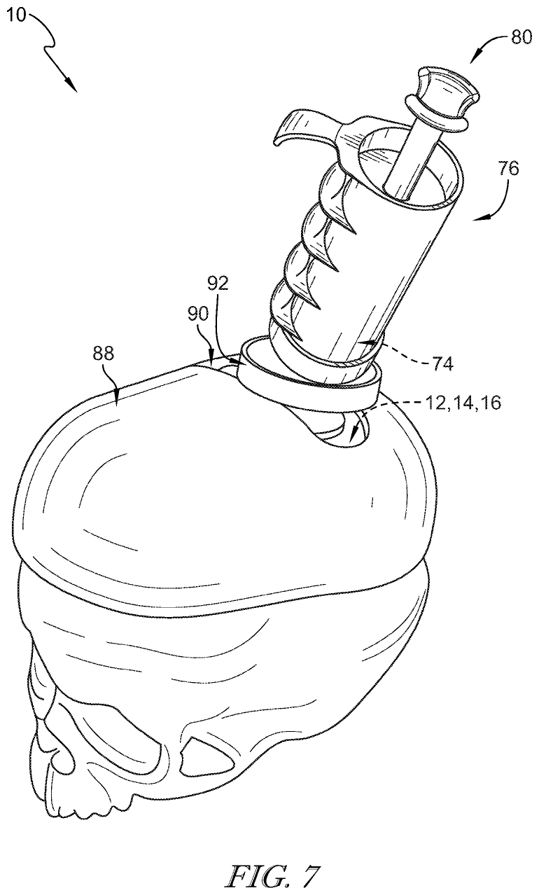

In some embodiments, the system 10 includes a guide helmet 88 and an attachment cap 92, as shown in FIG. 7. The guide helmet 88 may be 3D printed using pre-operative CT scans to match the curvature of the patient's skull. The position of the lever arm 64 and the ferromagnetic component 66 underneath the skin is marked on the guide helmet 88 via an opening 90 in the guide helmet 88. The attachment cap 92 is fixed to the guide helmet 88 at the opening 90. The guide helmet 88 allows the user to situate the external activator 76 over the location of the lever arm 64 and the ferromagnetic component 66 to ensure the magnetic force is applied to the ferromagnetic component 66. The attachment cap 92 provides stability to the user while using the external activator 76 and applying the external force to the plunger 80.

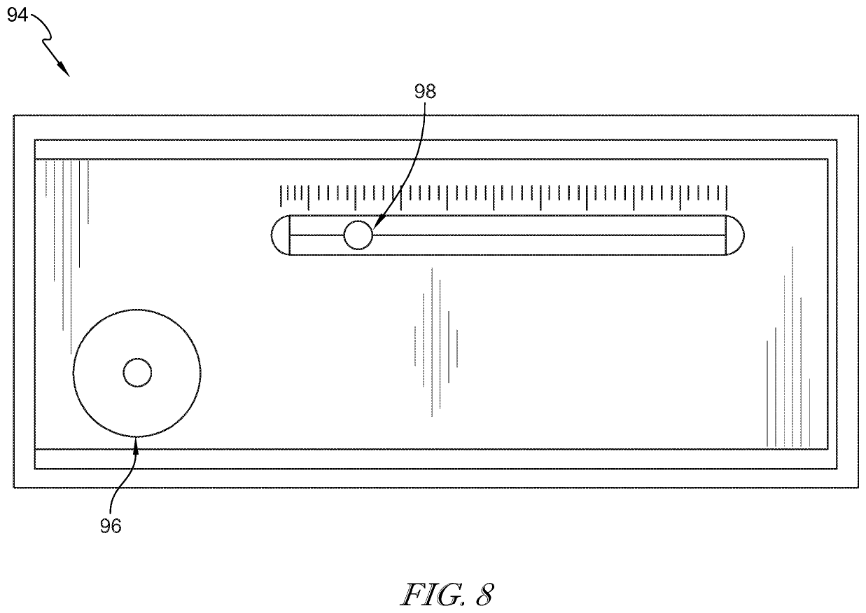

In some embodiments, the system 10 includes a measuring gauge 94, as shown in FIG. 8. The measuring gauge 94 allows the user to determine the distraction distance between the base members 18, 20. The measuring gauge 94 includes a first magnetic ball 96 and a second magnetic ball 98. The first magnetic ball 96 is arranged in a socket of the measuring gauge 94, and the second magnetic ball 98 is positioned in a liner track of the measuring gauge 94. As the measuring gauge 94 is held up to the skull, the first magnetic ball 96 centers on the magnetic field of the ferromagnetic component 66 in the lever arm 64, and the second magnetic ball 98 in the linear track moves along the liner track to align with a ferromagnetic component 35 included in the mobile base member 20. The location of the second magnetic ball 98 along the liner track allows the user to gauge the distance between the base members 18, 20.

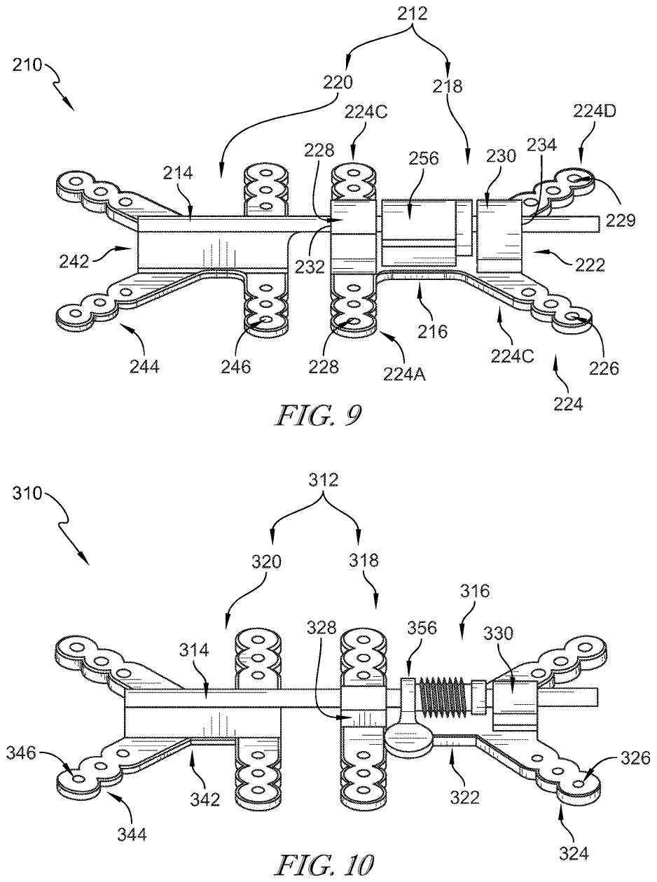

Another embodiment of a magnetically actuated cranial distraction system 210 in accordance with the present disclosure is shown in FIG. 9. The magnetically actuated cranial distraction system 210 is substantially similar to the magnetically actuated cranial distraction system 10 shown in FIGS. 1-4 and described herein. Accordingly, similar reference numbers in the 200 series indicate features that are common between the magnetically actuated cranial distraction system 10 and the magnetically actuated cranial distraction system 210. The description of the magnetically actuated cranial distraction system 10 is incorporated by reference to apply to the magnetically actuated cranial distraction system 210, except in instances when it conflicts with the specific description and the drawings of the magnetically actuated cranial distraction system 210.

As shown in FIG. 9, a bioresorbable distractor device 212 includes a first base member 218 and a second base member 220. The first base member 218 includes a base 222, a plate 224, and a plurality of fasteners 226. Illustratively, the plate 224 includes a first plate 224A, a second plate 224B, a third plate 224C, and a fourth plate 244D. The plates 224A, 224C extend outwardly away from the base 222 from a first side thereof, and the plates 224B, 244D extend outwardly away from the base 222 from a second side thereof opposite the first side. Each plate 224A, 224B, 224C, 244D is formed to include a plurality of fastener-receiver holes 229 extending therethrough to receive the plurality of fasteners 226 therein. The first base member 218 further comprises a first support bracket 228 and a second support bracket 230 that each extends upwardly away from the base 222. The first support bracket 228 is formed to include a first through hole 232 extending axially therethrough, and the second support bracket 230 is formed to include a second through hole 234 extending axially therethrough.

The second base member 220 includes a base 242, a plate 244, and a plurality of fasteners 246, as shown in FIG. 9. Illustratively, the plate 244 and the plurality of fasteners 246 are the same as the plate 224 and the plurality of fasteners 226. The shape of the plates 224A, 224B, 224C, 244D, 244 allow the base members 218, 220 to match a curvature of the patient's skull.

The system 210 includes a guide rod 214 coupled with the base 242 of the second base member 220, as shown in FIG. 9. The guide rod 214 extends through the through holes 232, 234 of the first base member 218. The system 210 further includes a distractor activation assembly 216. The distractor activation assembly 216 includes an activation lever 256 that rotates about an axis in response to a magnetic force.

Another embodiment of a magnetically actuated cranial distraction system 310 in accordance with the present disclosure is shown in FIG. 10. The magnetically actuated cranial distraction system 310 is substantially similar to the magnetically actuated cranial distraction system 10 shown in FIGS. 1-4 and the magnetically actuated cranial distraction system 210 shown in FIG. 9. Accordingly, similar reference numbers in the 300 series indicate features that are common between the magnetically actuated cranial distraction system 10, the magnetically actuated cranial distraction system 210, and the magnetically actuated cranial distraction system 310. The description of the magnetically actuated cranial distraction system 10, 210 is incorporated by reference to apply to the magnetically actuated cranial distraction system 310, except in instances when it conflicts with the specific description and the drawings of the magnetically actuated cranial distraction system 310.

A bioresorbable distractor device 312 includes a first base member 318 and a second base member 320, as shown in FIG. 10. The first base member 318 includes a base 322, a plate 324, and a plurality of fasteners 326. The first base member 318 further comprises a first support bracket 328 and a second support bracket 330 that each extend upwardly away from the base 322. The second base member 320 includes a base 342, a plate 344, and a plurality of fasteners 346, as shown in FIG. 10.

The system 310 includes a guide rod 314 coupled with the base 342 of the second base member 320 and a distractor activation assembly 316, as shown in FIG. 10. The distractor activation assembly 316 includes an activation lever 356 that rotates about an axis in response to a magnetic force. Compared to the activation lever 256 of the magnetically actuated cranial distraction system 210, the activation lever 356 of the magnetically actuated cranial distraction system 310 is more rounded.

Another embodiment of a magnetically actuated cranial distraction system 410 in accordance with the present disclosure is shown in FIG. 11. The magnetically actuated cranial distraction system 410 is substantially similar to the magnetically actuated cranial distraction system 10 shown in FIGS. 1-4, the magnetically actuated cranial distraction system 210 shown in FIG. 9, and the magnetically actuated cranial distraction system 310 shown in FIG. 10. Accordingly, similar reference numbers in the 400 series indicate features that are common between the magnetically actuated cranial distraction system 10, the magnetically actuated cranial distraction system 210, the magnetically actuated cranial distraction system 310, and the magnetically actuated cranial distraction system 410. The description of the magnetically actuated cranial distraction system 10, 210, 310 is incorporated by reference to apply to the magnetically actuated cranial distraction system 410, except in instances when it conflicts with the specific description and the drawings of the magnetically actuated cranial distraction system 410. Compared to the magnetically actuated cranial distraction system 210, 310, a plate 424 of a first base member 418 and a plate 444 of a second base member 420 are more compact. The system 410 includes a guide rod 414 coupled with a base 442 of the second base member 420 and a distractor activation assembly 416. The distractor activation assembly 416 includes an activation lever 456 that rotates about an axis in response to a magnetic force.

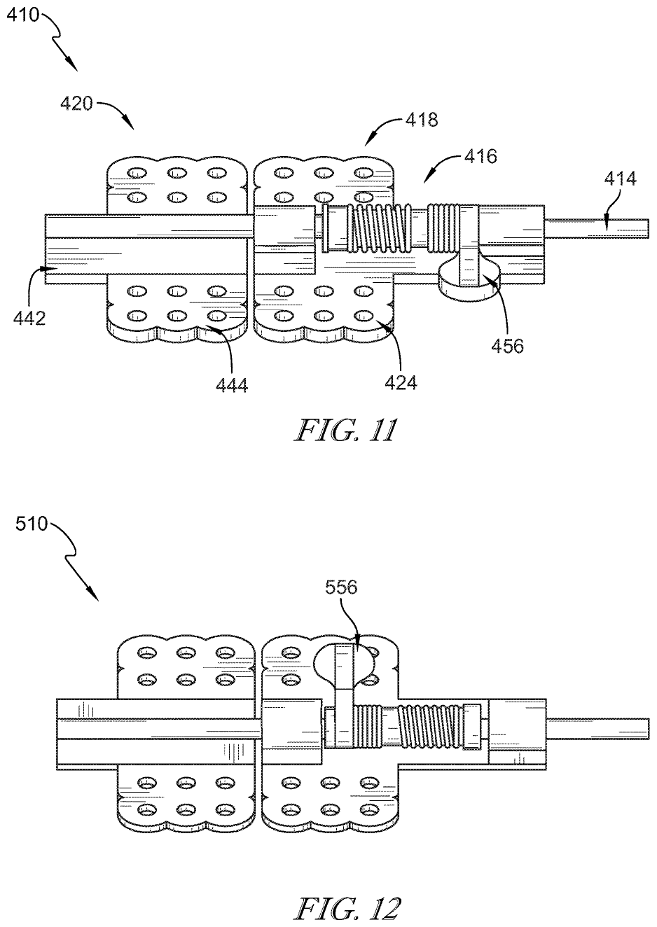

Another embodiment of a magnetically actuated cranial distraction system 510 in accordance with the present disclosure is shown in FIG. 12. The magnetically actuated cranial distraction system 510 is substantially similar to the magnetically actuated cranial distraction system 10 shown in FIGS. 1-4, the magnetically actuated cranial distraction system 210 shown in FIG. 9, the magnetically actuated cranial distraction system 310 shown in FIG. 10, and the magnetically actuated cranial distraction system 410 shown in FIG. 11. Accordingly, similar reference numbers in the 500 series indicate features that are common between the magnetically actuated cranial distraction system 10, the magnetically actuated cranial distraction system 210, the magnetically actuated cranial distraction system 310, the magnetically actuated cranial distraction system 410, and the magnetically actuated cranial distraction system 510. The description of the magnetically actuated cranial distraction system 10, 210, 310, 410 is incorporated by reference to apply to the magnetically actuated cranial distraction system 510, except in instances when it conflicts with the specific description and the drawings of the magnetically actuated cranial distraction system 510. Compared to the magnetically actuated cranial distraction system 410, the magnetically actuated cranial distraction system 510 is identical expect for the orientation of an activation lever 556 has been flipped.

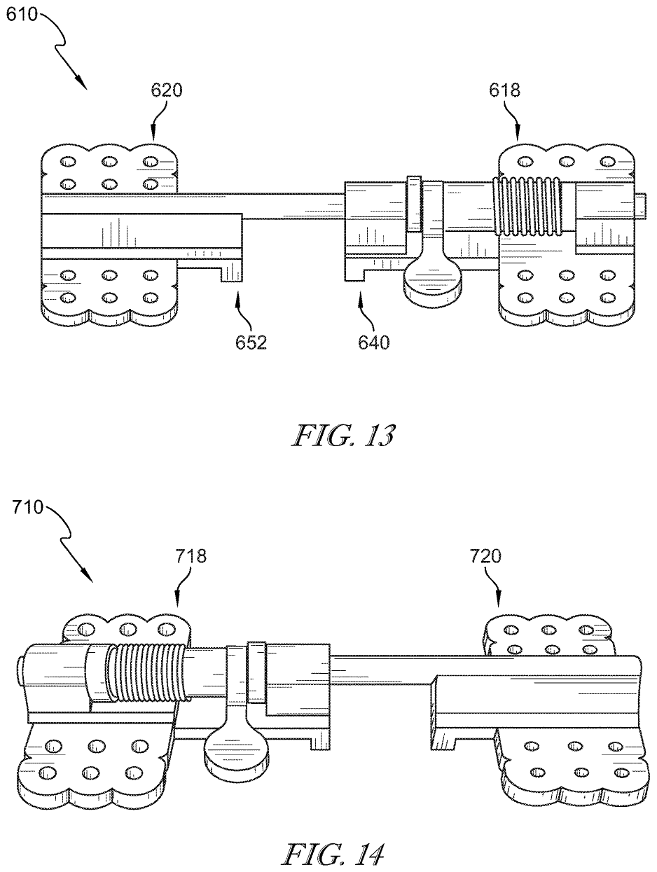

Another embodiment of a magnetically actuated cranial distraction system 610 in accordance with the present disclosure is shown in FIG. 13. The magnetically actuated cranial distraction system 610 is substantially similar to the magnetically actuated cranial distraction system 10 shown in FIGS. 1-4, the magnetically actuated cranial distraction system 210 shown in FIG. 9, the magnetically actuated cranial distraction system 310 shown in FIG. 10, the magnetically actuated cranial distraction system 410 shown in FIG. 11, and the magnetically actuated cranial distraction system 510 shown in FIG. 12. Accordingly, similar reference numbers in the 600 series indicate features that are common between the magnetically actuated cranial distraction system 10, the magnetically actuated cranial distraction system 210, the magnetically actuated cranial distraction system 310, the magnetically actuated cranial distraction system 410, the magnetically actuated cranial distraction system 510, and the magnetically actuated cranial distraction system 610. The description of the magnetically actuated cranial distraction system 10, 210, 310, 410, 510 is incorporated by reference to apply to the magnetically actuated cranial distraction system 610, except in instances when it conflicts with the specific description and the drawings of the magnetically actuated cranial distraction system 610.

As compared to the magnetically actuated cranial distraction system 210, 310, 410, 510, a first base member 618 and a second base member 620 are each formed to include a lip 640, 652 extending downwardly from the base member 618, 620. The lip 640, 652 is configured to position the base member 618, 620 relative to the cranial bone segment while the base member 618, 620 is being fixed to the cranial bone segment.

Another embodiment of a magnetically actuated cranial distraction system 710 in accordance with the present disclosure is shown in FIG. 14. The magnetically actuated cranial distraction system 710 is substantially similar to the magnetically actuated cranial distraction system 10 shown in FIGS. 1-4, the magnetically actuated cranial distraction system 210 shown in FIG. 9, and the magnetically actuated cranial distraction system 310 shown in FIG. 10, the magnetically actuated cranial distraction system 410 shown in FIG. 11, the magnetically actuated cranial distraction system 510 shown in FIG. 12, and the magnetically actuated cranial distraction system 610 shown in FIG. 13. Accordingly, similar reference numbers in the 700 series indicate features that are common between the magnetically actuated cranial distraction system 10, the magnetically actuated cranial distraction system 210, the magnetically actuated cranial distraction system 310, the magnetically actuated cranial distraction system 410, the magnetically actuated cranial distraction system 510, and the magnetically actuated cranial distraction system 610. The description of the magnetically actuated cranial distraction system 10, 210, 310, 410, 510, 610 is incorporated by reference to apply to the magnetically actuated cranial distraction system 710, except in instances when it conflicts with the specific description and the drawings of the magnetically actuated cranial distraction system 710. Compared to the magnetically actuated cranial distraction system 610, the magnetically actuated cranial distraction system 710 is identical, except that a first base member 718 and a second base member 720 switched orientations with one another.

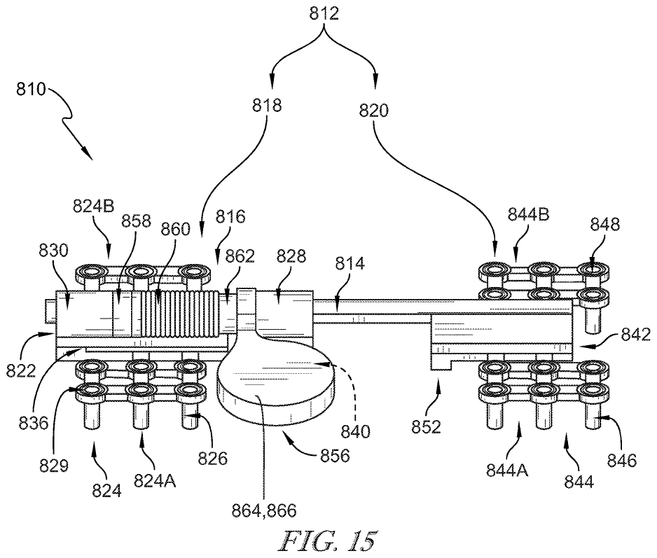

Another embodiment of a magnetically actuated cranial distraction system 810 in accordance with the present disclosure is shown in FIG. 15. The magnetically actuated cranial distraction system 810 is substantially similar to the magnetically actuated cranial distraction system 10 shown in FIGS. 1-4, the magnetically actuated cranial distraction system 210 shown in FIG. 9, the magnetically actuated cranial distraction system 310 shown in FIG. 10, the magnetically actuated cranial distraction system 410 shown in FIG. 11, the magnetically actuated cranial distraction system 510 shown in FIG. 12, the magnetically actuated cranial distraction system 610 shown in FIG. 13, and the magnetically actuated cranial distraction system 710 shown in FIG. 14. Accordingly, similar reference numbers in the 800 series indicate features that are common between the magnetically actuated cranial distraction system 10, the magnetically actuated cranial distraction system 210, the magnetically actuated cranial distraction system 310, the magnetically actuated cranial distraction system 410, the magnetically actuated cranial distraction system 510, the magnetically actuated cranial distraction system 610, and the magnetically actuated cranial distraction system 710. The description of the magnetically actuated cranial distraction system 10, 210, 310, 410, 510, 610, 710 is incorporated by reference to apply to the magnetically actuated cranial distraction system 810, except in instances when it conflicts with the specific description and the drawings of the magnetically actuated cranial distraction system 810.

The magnetically actuated cranial distraction system 810 includes a bioresorbable distractor device 812, as shown in FIG. 15. The bioresorbable distractor device 812 includes a first base member 818 and a second base member 820. The first base member 818 includes a base 822, a plate 824, and a plurality of fasteners 826. Illustratively, the plate 824 includes a first plate 824A and a second plate 824B. Each plate 824A, 824B is formed to include a plurality of fastener-receiver holes 829 extending therethrough to receive the plurality of fasteners 826 therein.

The first base member 818 further includes a first support bracket 828 and a second support bracket 830, as shown in FIG. 15. The first support bracket 828 and the second support bracket 830 extend upwardly away from the base 822. The base 822 is formed to include a first groove 836 on the first side thereof and a second groove on the second side thereof opposite the first side. The base 822 is further formed to include a lip 840 extending downwardly away from the first support bracket 828.

The second base member 820 includes a base 842, a plate 844, and a plurality of fasteners 846, as shown in FIG. 15. Illustratively, the plate 844 includes a first plate 844A and a second plate 844B. Each plate 844A, 844B is formed to include a plurality of fastener-receiver holes 848 extending therethrough to receive the plurality of fasteners 846 therein. The second base member 820 is formed to include a lip 852 extending downwardly away from the base 842.

The guide rod 814 extends through each of the second support bracket 830 of the first base member 818 and the first support bracket 828 of the first base member 818, as shown in FIG. 15. The guide rod 814 is fixed to the base 842 of the second base member 820.

The distractor activation assembly 816 includes an activation lever 856, a guide barrel 858, and a connection member 860, as shown in FIG. 15. The activation lever 856 rotates about an axis in response to a magnetic force. The activation lever 856 includes a lever barrel 862, a lever arm 864, and a ferromagnetic component 866. As compared to the activation lever 56, as shown in FIG. 1, the activation lever 856 includes a relatively larger lever arm 864 to accommodate a larger ferromagnetic component 866 therein.

The following numbered clauses include embodiments that are contemplated and non-limiting:
Clause 1. A magnetically actuated cranial distraction system including a distractor device configured to induce cranial bone growth, the distractor device including a fixed base member and a mobile base member, the fixed base member and the mobile base member are configured to be attached to cranial bone segments adjacent one another, a guide rod extending between the fixed base member and the mobile base member, the mobile base member configured to selectively move with the guide rod relative to the fixed base member, and a magnetically actuated ratchet coupled to the guide rod and configured to selectively move the mobile base member away from the fixed base member in response to a magnetic force being applied to the magnetically actuated ratchet.
Clause 2. The system of clause 1, of any one other suitable clause, or any suitable combination of clauses, wherein, upon the magnetic force being applied to the magnetically actuated ratchet, a portion of the magnetically actuated ratchet rotates about an axis and drives axial movement of the guide rod along the axis so that the mobile base member moves axially away from the fixed base member as the guide rod moves along the axis thereby distracting the cranial bone segment associated with the mobile base member from the cranial bone segment associated with the fixed base member.
Clause 3. The system of clause 2, of any one other suitable clause, or any suitable combination of clauses, wherein the magnetically actuated ratchet includes an activation lever, a guide barrel, and a connection member, the guide rod extends through the activation lever and the guide barrel, and the connection member extends between the activation lever and the guide barrel to transfer rotational energy from the activation lever to the guide barrel to cause the guide barrel to rotate about the axis with the activation lever.
Clause 4. The system of clause 3, of any one other suitable clause, or any suitable combination of clauses, wherein, in response to the magnetic force, the activation lever rotates about the axis in a first direction to cause the guide barrel to rotate about the axis therewith in the first direction and drive axial movement of the guide rod along the axis.
Clause 5. The system of clause 3, of any one other suitable clause, or any suitable combination of clauses, wherein the activation lever includes a lever barrel, a lever arm extending away from the lever barrel, and a ferromagnetic component included in the lever arm.
Clause 6. The system of clause 5, of any one other suitable clause, or any suitable combination of clauses, wherein the ferromagnetic component is spaced apart from the axis.
Clause 7. The system of clause 5, of any one other suitable clause, or any suitable combination of clauses, wherein the activation lever further includes a rotational blocker extending away from the lever barrel in a direction opposite the lever arm, and wherein the rotational blocker is configured to engage the fixed base member to block over rotation of the activation lever about the axis.
Clause 8. The system of clause 5, of any one other suitable clause, or any suitable combination of clauses, wherein the connection member comprises a wrap spring that extends around each of the lever barrel of the activation lever and the guide barrel to selectively couple the lever barrel to the guide barrel in response to rotation of the lever barrel about the axis in a first direction.
Clause 9. The system of clause 8, of any one other suitable clause, or any suitable combination of clauses, wherein the connection member decouples the lever barrel and the guide barrel from one another in response to rotation of the lever barrel about the axis in a second direction opposite the first direction.
Clause 10. The system of clause 3, of any one other suitable clause, or any suitable combination of clauses, wherein the fixed base member includes a base, a plate extending outwardly from the base and formed to include a plurality of fastener-receiver holes therein, and a plurality of fasteners configured to extend through the plurality of fastener-receiver holes to attach the fixed base member to the cranial bone segment.
Clause 11. The system of clause 10, of any one other suitable clause, or any suitable combination of clauses, wherein the fixed base member further includes a first support bracket and a second support bracket, the first support bracket and the second support bracket each extend upwardly away from the base of the fixed base member in spaced apart relation to one another, and wherein the first support bracket is formed to include a first through hole extending axially therethrough and the second support bracket is formed to include a second through hole extending axially therethrough.
Clause 12. The system of clause 11, of any one other suitable clause, or any suitable combination of clauses, wherein the activation lever, the connection member, and the guide barrel are located axially between the first support bracket and the second support bracket such that the first through hole of the first support bracket, a hole of the activation lever, a hole of the guide barrel, and the second through hole of the second support bracket are aligned with one another along the axis to receive the guide rod therein.
Clause 13. The system of clause 10, of any one other suitable clause, or any suitable combination of clauses, wherein the fixed base member includes a lip extending downwardly away from the base, and wherein the lip is configured to position the fixed base member relative to the cranial bone segment.
Clause 14. The system of clause 1, of any one other suitable clause, or any suitable combination of clauses, wherein the guide rod is fixed to the mobile base member so that the guide rod and the mobile base member axially translate with one another relative to the fixed base member.
Clause 15. The system of clause 1, of any one other suitable clause, or any suitable combination of clauses, wherein the mobile base member includes a base, a plate extending outwardly from the base and formed to include a plurality of fastener-receiver holes therein, and a plurality of fasteners configured to extend through the plurality of fastener-receiver holes to fix the mobile base member to the cranial bone segment.
Clause 16. The system of clause 15, of any one other suitable clause, or any suitable combination of clauses, wherein the base of the mobile base member is formed to include a hole extending axially therethrough to receive the guide rod therein, and wherein the guide rod is fixed to the mobile base member so that the mobile base member and the guide rod axially translate with one another.
Clause 17. The system of clause 1, of any one other suitable clause, or any suitable combination of clauses, wherein the distractor device, the guide rod, and the magnetically actuated ratchet are each configured to be implanted so that each of the distractor device, the guide rod, and the magnetically actuated ratchet are entirely internal to skin of a patient.
Clause 18. The system of clause 1, of any one other suitable clause, or any suitable combination of clauses, wherein the fixed base member and the mobile base member each comprise bioresorbable material.
Clause 19. The system of clause 18, of any one other suitable clause, or any suitable combination of clauses, wherein the bioresorbable material is a magnesium-based alloy of magnesium, gallium, and zinc.
Clause 20. The system of clause 1, of any one other suitable clause, or any suitable combination of clauses, further comprising an external activator configured to apply the magnetic force to the magnetically actuated ratchet, and wherein the external activator includes a permanent magnet that applies the magnetic force to the magnetically actuated ratchet in response to being proximate the magnetically actuated ratchet.
Clause 21. A magnetically actuated cranial distraction system comprising a bioresorbable distractor device configured to induce cranial bone growth, the distractor device including a first base member and a second base member, the first base member and the second base member are configured to be attached to cranial bone segments adjacent one another along an axis, a threaded rod extending along the axis between the first base member and the second base member, the second base member being configured to selectively move with the threaded rod along the axis relative to the first base member, the first base member being fixed in place, and a distractor activation assembly configured to selectively move the second base member away from the first base member along the axis, the distractor activation assembly including an activation lever, a threaded barrel formed to include a threaded hole extending axially therethrough, and a connection member, the threaded rod extends through the activation lever and through the threaded barrel to mate with the threaded hole of the threaded barrel, and the connection member extends between the activation lever and the threaded barrel to transfer rotational energy in a first direction from the activation lever to the threaded barrel to cause the threaded barrel to rotate about the axis with the activation lever in the first direction, wherein, in response to a magnetic force being applied to the activation lever, the activation lever rotates about the axis in the first direction to cause the threaded barrel to rotate about the axis therewith to drive axial movement of the threaded rod along the axis so that the second base member moves axially away from the first base member as the threaded rod moves along the axis thereby distracting the cranial bone segment associated with the second base member from the cranial bone segment associated with the first base member.
Clause 22. The system of clause 21, of any one other suitable clause, or any suitable combination of clauses, wherein the activation lever includes a lever barrel formed to include a hole extending axially therethrough, a lever arm extending away from the lever barrel, and a ferromagnetic component included in the lever arm, and wherein the threaded rod extends through the hole of the lever barrel.
Clause 23. The system of clause 22, of any one other suitable clause, or any suitable combination of clauses, wherein the ferromagnetic component is spaced apart from the axis.
Clause 24. The system of clause 22, of any one other suitable clause, or any suitable combination of clauses, wherein the activation lever further includes a rotational blocker extending away from the lever barrel in a direction opposite the lever arm, and wherein the rotational blocker is configured to engage the first base member to block over rotation of the activation lever about the axis in the first direction.
Clause 25. The system of clause 22, of any one other suitable clause, or any suitable combination of clauses, wherein the connection member comprises a wrap spring that extends around each of the lever barrel of the activation lever and the threaded barrel to selectively couple the lever barrel to the threaded barrel in response to rotation of the lever barrel about the axis in the first direction.
Clause 26. The system of clause 22, of any one other suitable clause, or any suitable combination of clauses, wherein the first base member includes a base, a plate extending outwardly from the base and formed to include a plurality of fastener-receiver holes therein, and a plurality of fasteners configured to extend through the plurality of fastener-receiver holes to attach the first base member to the cranial bone segment.

Clause 27. The system of clause 26, of any one other suitable clause, or any suitable combination of clauses, wherein the first base member further includes a first support bracket and a second support bracket, the first support bracket and the second support bracket each extends upwardly away from the base of the first base member in spaced apart relation to one another, and wherein the first support bracket is formed to include a first through hole extending axially therethrough and the second support bracket is formed to include a second through hole extending axially therethrough.
Clause 28. The system of clause 27, of any one other suitable clause, or any suitable combination of clauses, wherein the lever barrel, the connection member, and the threaded barrel are located axially between the first support bracket and the second support bracket such that the first through hole of the first support bracket, the hole of the lever barrel, the threaded hole of the threaded barrel, and the second through hole of the second support bracket are aligned with one another along the axis to receive the threaded rod therein.
Clause 29. The system of clause 26, of any one other suitable clause, or any suitable combination of clauses, wherein the first base member includes a lip extending downwardly away from the base, and wherein the lip is configured to position the first base member relative to the cranial bone segment.
Clause 30. The system of clause 21, of any one other suitable clause, or any suitable combination of clauses, wherein the second base member includes a base, a plate extending outwardly from the base and formed to include a plurality of fastener-receiver holes therein, and a plurality of fasteners configured to extend through the plurality of fastener-receiver holes to attach the second base member to the cranial bone segment.
Clause 31. The system of clause 30, of any one other suitable clause, or any suitable combination of clauses, wherein the base of the second base member is formed to include a threaded hole extending axially therethrough to receive the threaded rod therein, and wherein the threaded rod is fixed to the second base member so that the second base member and the threaded rod axially translate with one another.

Clause 32. The system of clause 21, of any one other suitable clause, or any suitable combination of clauses, wherein the first base member and the second base member each comprise bioresorbable material, and wherein the bioresorbable material is a magnesium-based alloy of magnesium, gallium, and zinc.
Clause 33. The system of clause 21, of any one other suitable clause, or any suitable combination of clauses, further comprising an external activator configured to apply the magnetic force to the activation lever, and wherein the external activator includes a permanent magnet that applies the magnetic force to the activation lever in response to being proximate the activation lever.
Clause 34. A method of distraction osteogenesis comprising providing a bioresorbable distractor device, a guide rod, and a magnetically actuated ratchet, the bioresorbable distractor device including a fixed base member and a mobile base member, attaching the fixed base member and the mobile base member to cranial bone segments, coupling the guide rod to each of the fixed base member, the mobile base member, and the magnetically actuated ratchet, applying a magnetic force to an activation lever of the magnetically actuated ratchet, in response to the magnetic force, rotating the activation lever of the magnetically actuated ratchet about an axis in a first direction, in response to rotation of the activation lever of the magnetically actuated ratchet about the axis, axially translating the guide rod relative to the fixed base member, and in response to axial translation of the guide rod, axially translating the mobile base member with the guide rod relative to the fixed base member.
Clause 35. The method of clause 34, of any one other suitable clause, or any suitable combination of clauses, further comprising, during the step of rotating the activation lever of the magnetically actuated ratchet, transferring rotational energy from the activation lever to a guide barrel of the magnetically actuated ratchet to cause the guide barrel to rotate about the axis in the first direction with the activation lever.
Clause 36. The method of clause 35, of any one other suitable clause, or any suitable combination of clauses, further comprising removing the magnetic force from the activation lever and, after the step of removing the magnetic force from the activation lever, rotating the activation lever about the axis in a second direction opposite the first direction.
Clause 37. The method of clause 36, of any one other suitable clause, or any suitable combination of clauses, further comprising, during the step of rotating the activation lever about the axis in the second direction opposite the first direction, maintaining a position of the guide barrel.
Clause 38. The method of clause 34, of any one other suitable clause, or any suitable combination of clauses, further comprising implanting the bioresorbable distractor device, the guide rod, and the magnetically actuated ratchet in a patient such that each of the bioresorbable distractor device, the guide rod, and the magnetically actuated ratchet are entirely located underneath skin of the patient.
Clause 39. A method comprising positioning a connection member on one of a guide barrel or an activation lever, aligning the guide barrel and the activation lever such that the connection member extends therebetween, positioning the guide barrel and the activation lever between a first support bracket and a second support bracket of a fixed base member, extending a guide rod through the first support bracket, the guide barrel, the activation lever, the second support bracket, and into a mobile base member, attaching the fixed base member and the mobile base member to cranial bone segments of a patient, positioning each of the connection member, the guide barrel, the activation lever, the fixed base member, the guide rod, and the mobile base member entirely underneath skin of the patient, applying a magnetic force to the activation lever from an area external to the patient, and moving the mobile base member away from the fixed base member in response to the magnetic force.
Clause 40. The method of clause 39, of any one other suitable clause, or any suitable combination of clauses, further comprising rotating the activation lever about an axis in a first direction in response to the magnetic force.
Clause 41. The method of clause 40, of any one other suitable clause, or any suitable combination of clauses, further comprising, in response to rotation of the activation lever about the axis, axially translating the guide rod relative to the fixed base member.
Clause 42. The method of clause 41, of any one other suitable clause, or any suitable combination of clauses, further comprising, in response to axial translation of the guide rod, moving the mobile base member with the guide rod away from the fixed base member.
Clause 43. The method of clause 39, of any one other suitable clause, or any suitable combination of clauses, further comprising resorbing the fixed base member and the mobile base member into the patient after a period of time.
Clause 44. A method of distraction osteogenesis comprising implanting a bioresorbable distractor device, a guide rod, and a distractor activation assembly in a patient such that each of the bioresorbable distractor device, the guide rod, and the distractor activation assembly are entirely underneath skin of the patient, positioning an external activator near the distractor activation assembly, the external activator located entirely external to the skin of the patient, in response to the external activator being proximate the distractor activation assembly, axially translating a mobile base member of the bioresorbable distractor device relative to a fixed base member of the bioresorbable distractor device, and distracting a cranial bone segment associated with the mobile base member from a cranial bone segment associated with the fixed base member.
Clause 45. The method of clause 44, of any one other suitable clause, or any suitable combination of clauses, further comprising, in response to the external activator being proximate the distractor activation assembly, rotating a portion of the distractor activation assembly about an axis in a first direction.
Clause 46. The method of clause 45, of any one other suitable clause, or any suitable combination of clauses, further comprising, in response to rotation of the portion of the distractor activation assembly, axially translating the guide rod relative to the fixed base member.
Clause 47. The method of clause 46, of any one other suitable clause, or any suitable combination of clauses, further comprising, in response to axial translation of the guide rod, axially translating the mobile base member of the bioresorbable distractor device with the guide rod relative to the fixed base member.
Clause 48. The method of clause 45, of any one other suitable clause, or any suitable combination of clauses, further comprising moving the external activator away from the distractor activation assembly.
Clause 49. The method of clause 48, of any one other suitable clause, or any suitable combination of clauses, further comprising, in response to moving the external activator away from the distractor activation assembly, rotating the portion of the distractor activation assembly about the axis in a second direction opposite the first direction.
Clause 50. The method of clause 44, of any one other suitable clause, or any suitable combination of clauses, further comprising resorbing the fixed base member and the mobile base member into the patient after a period of time.

1-50. (canceled)

51. A magnetically actuated cranial distraction system comprising:
a distractor device configured to induce cranial bone growth, the distractor device including a fixed base member and a mobile base member, the fixed base member and the mobile base member are configured to be attached to cranial bone segments adjacent one another,
a guide rod extending between the fixed base member and the mobile base member, the mobile base member configured to selectively move with the guide rod relative to the fixed base member, and
a distractor activation assembly coupled to the guide rod and configured to selectively move the mobile base member away from the fixed base member in response to a magnetic force being applied to the distractor activation assembly.

52. The system of claim 51, wherein, upon the magnetic force being applied to the distractor activation assembly, a portion of the distractor activation assembly rotates about an axis and drives axial movement of the guide rod along the axis so that the mobile base member moves axially away from the fixed base member as the guide rod moves along the axis thereby distracting the cranial bone segment associated with the mobile base member from the cranial bone segment associated with the fixed base member.

53. The system of claim 52, wherein the distractor activation assembly includes an activation magnet unit, a guide barrel, and a connection member, the guide rod extends through the guide barrel, and the connection member extends between the activation magnet unit and the guide barrel to transfer rotational energy from the activation magnet unit to the guide barrel to cause the guide barrel to rotate about the axis.

54. The system of claim 53, wherein the activation magnet unit includes a ferromagnetic component that is spaced apart from the axis.

55. The system of claim 53, wherein the fixed base member includes a base, a first support bracket, and a second support bracket, the first support bracket and the second support bracket each extend upwardly away from the base in spaced apart relation to one another, and wherein the first support bracket is formed to include a first through hole extending axially therethrough and the second support bracket is formed to include a second through hole extending axially therethrough.

56. The system of claim 55, wherein the guide barrel is located axially between the first support bracket and the second support bracket such that the first through hole of the first support bracket, a hole of the guide barrel, and the second through hole of the second support bracket are aligned with one another along the axis to receive the guide rod therein.

57. The system of claim 56, wherein the hole of the guide barrel is formed to include internal threads, and wherein the guide rod is formed to include external threads that mate with the internal threads of the guide barrel.

58. The system of claim 53, further comprising a cover coupled to the fixed base member and positioned over the guide barrel and the connection member.

59. The system of claim 51, wherein the fixed base member includes a base, a first support bracket, and a second support bracket, the first support bracket and the second support bracket each extend upwardly away from the base in spaced apart relation to one another, and wherein the first support bracket is formed to include a first through hole extending axially therethrough and the second support bracket is formed to include a second through hole extending axially therethrough.

60. The system of claim 59, wherein the fixed base member further includes a lip extending downwardly away from the base, and wherein the lip is configured to position the fixed base member relative to the cranial bone segment.

61. The system of claim 59, wherein the mobile base member includes a base formed to include a hole extending axially therethrough to receive the guide rod therein, the hole being aligned with the first through hole and the second through hole of the fixed base member, and wherein the guide rod is fixed to the mobile base member so that the mobile base member and the guide rod axially translate with one another.

62. The system of claim 51, further comprising an external activator configured to apply the magnetic force to the distractor activation assembly, and wherein the external activator includes a body and a permanent magnet located within the body and configured to move within the body toward and away from the distractor activation assembly.

63. A method comprising:
providing a distractor device, a guide rod, and a distractor activation assembly, the distractor device including a fixed base member and a mobile base member,
attaching the fixed base member and the mobile base member to cranial bone segments,
coupling the guide rod to each of the fixed base member, the mobile base member, and the distractor activation assembly,
applying a magnetic force to an activation magnet unit of the distractor activation assembly,
in response to the magnetic force, rotating the activation magnet unit of the distractor activation assembly,
in response to rotation of the activation magnet unit of the distractor activation assembly, axially translating the guide rod relative to the fixed base member along an axis, and
in response to axial translation of the guide rod, axially translating the mobile base member with the guide rod relative to the fixed base member.

64. The method of claim 63, further comprising, during the step of rotating the activation magnet unit of the distractor activation assembly, transferring rotational energy from the activation magnet unit to a guide barrel of the distractor activation assembly to cause the guide barrel to rotate about the axis.

65. The method of claim 63, further comprising implanting the distractor device, the guide rod, and the distractor activation assembly in a patient such that each of the distractor device, the guide rod, and the distractor activation assembly are entirely located underneath skin of the patient.

66. The method of claim 63, further comprising positioning a guide barrel of the distractor activation assembly between a first support bracket and a second support bracket of the fixed base member.

67. The method of claim 66, further comprising extending the guide rod through a first through hole of the first support bracket, a threaded hole of the guide barrel, a second through hole of the second support bracket, and a threaded hole of the mobile base member.

68. The method of claim 66, further comprising coupling a cover to the fixed base member over the guide barrel.

69. The method of claim 63, wherein applying the magnetic force to the activation magnet unit of the distractor activation assembly includes applying the magnetic force to the activation magnet unit from an area external to the patient.

70. The method of claim 63, wherein applying the magnetic force to the activation magnet unit of the distractor activation assembly includes positioning an external activator near the activation magnet unit and moving a permanent magnet within the external activator toward the activation magnet unit.
